(19) United States
(12) Patent Application Publication
(10) Pub. No.: US 2024/0376531 A1
(43) Pub. Date: Nov. 14, 2024

(54) Target Initiation and Amplification of Long DNA with Nuclease and Replisome Enzymes

(71) Applicant: New England Biolabs, Inc., Ipswich, MA (US)

(72) Inventors: Nathan Tanner, West Newbury, MA (US); Dillon B. Nye, Somerville, MA (US)

(73) Assignee: New England Biolabs, Inc., Ipswich, MA (US)

(21) Appl. No.: 18/563,125

(22) PCT Filed: Jun. 9, 2022
(86) PCT No.: PCT/US22/32777

§ 371 Date: Nov. 21, 2023

Related U.S. Application Data
(60) Provisional application No. 63/208,534, filed on Jun. 9, 2021.
(60) Provisional application No. 63/208,621, filed on Jun. 9, 2021.

(51) Int. Cl. C12Q 1/6844

(52) U.S. Cl. CPC C12Q 1/6844

(57) ABSTRACT

Compositions, methods and kits are provided for long range amplification of nucleic acid sequences in vitro. This includes a site-specific or sequence-specific nick in one strand of a duplex to initiate strand displacement linear amplification without the need for primers. Preferably, modified components of a T7 replisome are used for the in vitro linear amplification reaction enabling copying of at least 300 bases and as much as 50 kb of the target nucleic acid.

### BACKGROUND

Myriad isothermal strategies have been developed to overcome the limitations of the polymerase chain reaction (PCR) for amplifying DNA. The limitations of PCR include the requirement for thermal denaturation and thermocycling where thermocycling requires specialized equipment and conditions vary according to the polymerase used. Moreover, thermocycling can damage target DNA introducing random and unspecified mutations in the DNA sequence. The length of amplicons produced by PCR using Taq polymerase shows significant variability even under carefully selected thermocycling conditions (Jia et al. Scientific Reports vol 4, Article No. 5737 (2014)). In addition, amplification of a long DNA is very time intensive. For example, amplification of a 50 kilobases (kb) fragment could take as long as 29 hours and the outcome could be uncertain as to whether any amplicons could be produced.

Isothermal or non-thermocycling amplification methods are alternatives to PCR and have found utility in molecular diagnostics where short target amplification is desirable for detection speed. Advances in long read DNA sequencing and synthetic biology make desirable the development of isothermal DNA amplification methods for amplifying DNA targets over 20 kb in length.

Phage T7 replisome is capable of long range copying of a DNA. A complex of polymerase, a processivity factor, and helicase mediates leading strand synthesis. Establishment of the complex requires an interaction of the C-terminal tail of the helicase with the polymerase. During synthesis the complex is stabilized by other interactions. The C-terminal tail also interacts with a distinct region of the polymerase to capture dissociating polymerase to increase the processivity to >17 kb. The lagging strand is synthesized discontinuously within a loop that forms and resolves during each cycle of Okazaki fragment synthesis (see FIG. 1A). The synthesis of a primer as well as the termination of a fragment signal loop resolution (Seung-Joo Lee, Curr Opin Chem Biol. 2011 October; 15 (5): 580-586). The T7 replisome is a dynamic macromolecular assembly of only four proteins: gp2.5, a homodimeric single-stranded DNA (ssDNA) binding protein; T7 gp4, a hexameric ATPase that functions as both a helicase and a primase; T7 gp5, a DNA polymerase; and Escherichia coli thioredoxin (Trx), which binds gp5 to increase the processivity of DNA synthesis (see FIG. 1B). The helicase gp4 encircles one strand of a double-stranded DNA (dsDNA) substrate and translocates in the 5′ to 3′ direction with the primase domain synthesizing RNA primers for lagging-strand synthesis, while the other strand is used as a template for continuous leading-strand synthesis by gp5. Because lagging-strand synthesis is discontinuous and requires additional proteins for maturation, this can reduce the amount of replicated DNA as does strand switching which is commonly observed in phage DNA replication. In vitro strand switching is undesirable because it results in bias in copy representation and uneven coverage of the target sequence. While the replicative potential of the T7 replisome is sufficient and appropriate for the needs of the virus, it is not optimal for genomic amplification in vitro.

One approach to amplification of whole genomic DNA is to use the T7 replisome and introduce random nicks into one strand of a DNA duplex to provide a convenient means of non-specific initiation or primer-specific initiation of genomic DNA amplification using a strand displacing polymerase-helicase complex. This approach can result in leading and lagging strand replication and associated artifacts associated with lagging strand synthesis.

There is a need for improvements in methods for reliable, controllable amplification of long targeted portions of the genome for use in sequencing platforms and synthetic biology applications that can utilize long DNA.

### SUMMARY

In general, a composition is provided that includes: a mesophilic nickase that is strand-specific, sequence-specific and/or site-specific and capable of multiple turnover kinetics, aT7 polymerase (exo-), a helicase, and a single-strand binding protein (SSBP) wherein the composition is free of primers. The helicase in the composition may lack primase activity or alternatively, the helicase has primase activity but the composition excludes rCTP and rATP. For example, the helicase may be a modified phage T7 gp4 helicase. The SSBP may be phage T7 (gp2.5) and Trx may be included.

In one embodiment, the nickase is sequence-specific, wherein the sequence-specific nickase is selected from the group consisting of a modified restriction endonuclease and a mesophilic argonaut having a guide DNA.

In another embodiment, the nickase is site-specific wherein the site-specific nickase is selected from the group consisting of endonuclease, III, endonuclease V or endonuclease VIII; formamidopyrimidine DNA glycosylase (Fpg) and RNaseHII.

The composition may include a DNA adaptor, where the adaptor includes a nickase specific recognition sequence or a sequence for hybridizing to a guide DNA, or a nick site selected from a ribonucleotide or a modified deoxyribonucleotide such as 8-oxoguanine.

The nickase, T7 polymerase (exo-), SSBP and the helicase may be lyophilized or immobilized on a matrix either separately or together.

Primers may be added to the composition in which case they may have a complementary sequence to the 3′ sequence adjacent to a nickase recognition sequence. The primers may include the nickase recognition sequence and contain a sequence for hybridizing to target DNA.

In general, a method is provided for in vitro long range linear amplification of target dsDNA. comprising: (a) combining the dsDNA with the composition as described above to produce a reaction mix in a reaction container; and (b) incubating the reaction mix under isothermal conditions to produce an amplification product.

In some embodiments, within the reaction mix: the target dsDNA is nicked by the nickase; the amplifying is by leading strand displacement by the DNA polymerase, wherein the individual strands of the dsDNA start at one nick site on one strand and terminate at the nick site on the second strand to produce daughter strands; and the daughter strands become nicked, thereby permitting rounds of nicking and strand displacement.

In general, a method is also provided for long range linear amplification of target dsDNA, which includes the following steps: (a) nicking a target DNA duplex by means of a strand or site-specific mesophilic nickase capable of multiple turnover kinetics; (b) amplifying by linear strand displacement with a strand displacing polymerase the individual strands of the duplex starting at one nick site on one strand and terminating at the nick site on the second strand to produce daughter strands; and (c) allowing the daughter strands to become nicked and permitting rounds of nicking and strand displacement.

Various embodiments of the method may have one or more of the following features: the nickase may be a modified restriction endonuclease having a recognition sequence of 5, 6 or 7 bases; the helicase may lack a primase or rCTP and rATP is excluded from the reaction mixture; and the polymerase may be a T7 polymerase (exo-).

One embodiment of the method may include the additional feature of hybridizing one or more primers to a DNA sequence that is the 3′ adjacent sequence to a nickase recognition sequence.

The primers may be designed to include a universal sequence and a nickase recognition sequence and permitting the universal sequence on the primer to hybridize to the target DNA.

The target sequence for amplification may have a length in the range of 300 bases to 50,000 bases.

Other features of the method may include performing two or more of steps (a), (b), and (c) of the method above in the same reaction vessel and/or (a)-(c) is performed in a one-step reaction, and/or performing the reactions at 37° C.

In another embodiment, a method for amplifying long target DNA in vitro includes: (a) nicking the target DNA on opposing strands with a sequence-specific or site-specific mesophilic nickase capable of multiple turnover kinetics; (b) replicating the leading strand of the duplex only with a T7 exo-polymerase in the presence of a helicase, SSBP and dNTPs; and (c) producing full length amplicons from a target DNA of at 300 bases to at least 50 kb.

In general, a kit is provided that includes: a sequence-specific or site-specific mesophilic nickase capable of multiple turnover kinetics, T7 polymerase (exo-), a SSBP, a helicase and dNTPs in a single container or in separate containers either in a buffer, immobilized on a matrix or lyophilized.

### DESCRIPTION OF FIGURES

FIG. 1A shows the continuous replication of DNA on a leading strand (101) and Okazaki fragments (103) produced from RNA primers on the lagging strand (102).

FIG. 1B shows T7 replisome complex as it replicates its genome in a host cell. DNA polymerase and DNA helicase are essential components of DNA replication. The helicase unwinds duplex DNA to provide single-stranded templates for DNA synthesis by the DNA polymerase. The T7 replisome generally terminates at an adjacent nick in duplex DNA. The components are labelled as follows: Trx (104), gp5 DNA polymerase (105), gp4 is RPD (106), ZBD (107), helicase (108) where (106) and (107) are the primase. The primer is labeled (109), terminal tail emerging from each of the 6 helicase subunits is (110) and the ssDNA binding protein gp2.5 is (111).

FIG. 2 shows a schematic representation of an in vitro method that relies on T7 polymerase (exo) in combination with a strand-specific and site-specific or sequence-specific nickase and other T7 replisome proteins to synthesize a targeted portion of a genomic DNA to produce an amplicon of a gene of interest that may be several kilobases in length. The template (112) is nicked at a sequence-specific target on one strand and the same sequence-specific target on the complementary strand. T7 replisome produces separated daughter templates (113) and daughter templates are nicked at the same sequence-specific target as the parent strands (115) and filled in (114) resulting in rounds of nicking and strand displacement synthesis (116). In this embodiment, the amplification product accumulates in a linear manner not exponentially hence it is referred to as linear amplification.

FIG. 3A-FIG. 3C show amplicons produced by strand displacement amplification after nicking with each of three different nickases under varying conditions including time and gp5 DNA polymerase or an exo-variant thereof in addition to the other three proteins that make up a replisome (gp2.5, Trx and helicase). FIG. 3D shows amplicons produced by strand displacement amplification after endonuclease V nicking of inosine in lambda DNA.

FIG. 3A shows that T7 replisome amplification is not dependent on any single nickase but rather any site-specific nick can enable long strand synthesis of target DNA. Agarose gel electrophoresis show amplicons produced by T7-SDA of lambda DNA with either the WT or delta 28 gp5 (exo-) T7 replisome and each of three different nickases: Nt.BbvCI, Nb.BbvCI or Nb.BssSI. “Nb” is bottom strand nickase while “Nt” is top strand nickase.

FIG. 3B and FIG. 3C show how the amplification method of FIG. 2 was used to produce a 7714 bp substrate with sequential Nb.BssSI recognition sequences (sequence-specific target). The results are shown on a gel over different incubation times varying from 15 minutes to 75 minutes. The amplicon was combined in reaction buffer (50 mM Tris acetate pH 7.9, 50 mM potassium acetate, 2 mM DTT, 3.5 mM dTTP, 1 mM other dNTPs) with 5 μM gp2.5 (dimer), 100 nM gp4 (hexamer), 200 U mL⁻¹ Sequenase 2.0 and 200 U mL⁻¹ of Nb.BssSI. Reactions were initiated by addition of magnesium acetate to 10 mM and incubated at 37° C. Aliquots were removed at various time points, quenched with 6×Purple Loading Dye (New England Biolabs, Ipswich, MA) and visualized using 1.0% agarose gel electrophoresis.

FIG. 3C provides quantitative values for the amplification product. 25 ng of 7 kb dsDNA substrate produced 2 μg (80×) of amplicon in 75 minutes. The T7 replisome has yielded as much as 300×amplification, with yield depending on time, reaction conditions and starting concentration of the target DNA.

FIG. 3D is a similar reaction to FIG. 3B except that the nicking is site specific instead of sequence specific where the site is deoxyinosine and the nickase is endonuclease V. Reactions were as above but with 200 U mL−1 of endonuclease V as the nicking enzyme.

FIG. 4A-FIG. 4D show coverage maps from nanopore sequencing data for T7 SDA reactions of various sized amplicons (0.3 kb-12.8 kb) of lambda DNA generated by the method shown in FIG. 2.

Each panel shows coverage maps of the 3′ and 5′ ends of the alignments, as well as full alignment produced by minimap2. Counts per total alignments reflect the coverage at each nucleotide divided by the number of alignments. At the top are diagrams showing the position and orientation of nicking recognition sites within the lambda phage genome, where lines above the horizontal correspond to anticipated top strand nicks, and those below to bottom strand nicks. The nicking enzyme and polymerase used in each panel are: (A) Nt.BbvCI and delta 28 gp5; (B) Nb.BbvCI and delta 28 gp5; (C) Nb.BssSI and delta 28 gp5.

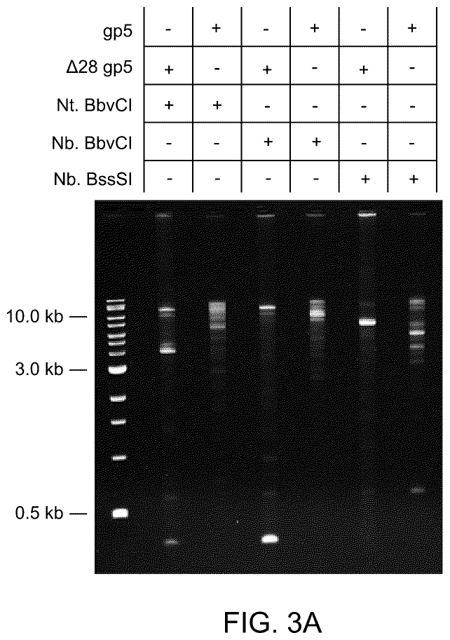

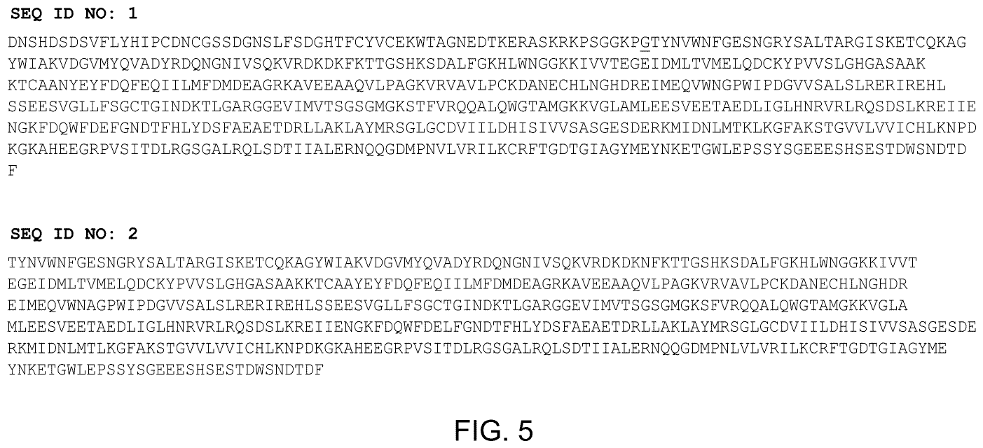

In each of FIG. 4A-FIG. 4D, the line at top of the histogram represents the recognition sequences for the nickase on top and bottom strands where linear amplification is initiated. Amplicons arise from regions between sequential top and bottom strand recognition sequences within the λ-phage genome. The size of amplicons is limited here by the arrangement of recognition sequences, and in general by the specificity of the nickase. In rare instances, the replisome passes through a nick site to generate a larger fragment as shown in the figure. 5′ ends are generated in the SDA reaction by the nicking enzyme and have lower heterogeneity compared to 3′ ends which result from polymerase activity. The 5′ coverage maps demonstrate that amplified dsDNA is generated by repeated cycles of nicking and strand-displacing DNA polymerization. Reactions with the exonuclease deficient T7 replisome show prominent regions of high coverage between sequential top and bottom-strand nicking recognition sites as expected from the amplification scheme of FIG. 2.

Genomic λ-phage DNA was incubated with components of the T7 replisome (5 μM dimer of gp2.5, 100 nM hexamer gp4, 200 U mL⁻¹ Sequenase 2.0) and 200 U mL⁻¹ Nt.BbvCI in reaction buffer (50 mM Tris acetate pH 7.9, 50 mM potassium acetate, 2 mM DTT, 3.5 mM dTTP, 1 mM other dNTPs). Reactions (50 μL) were initiated with 10 mM magnesium acetate, incubated at 37° C. for 3 hours, and quenched with a mixture of 20 mM EDTA and Proteinase K.

Amplified DNA fragments were sequenced by Oxford Nanopore Sequencing. Sequencing reads were filtered by size and aligned to the λ-phage genome sequence using the minimap2 algorithm. Coverage maps were generated from the size-filtered sequencing reads.

FIG. 5 describes the sequences for wild type gP4 sequence of M64G gp4 lacking the N-terminal methionine (SEQ ID NO:1) and an example of a primase deletion mutant where the N-terminal zinc finger has been deleted for use as a strand displacing polymerases herein. (SEQ ID NO:2).

### DETAILED DESCRIPTION

Methods and compositions are provided herein for linear long range strand displacement amplification (SDA) of targeted dsDNA sequences (e.g. regions of a genome) by means of a modified T7 replisome (T7-SDA) that relies on a site-specific or sequence-specific nickase to initiate strand displacement on the leading strand only. Primers are not required for nick-initiated amplification.

The nickases cut the bottom or top strands of the target DNA at specific sites or sequences to enable SDA from these nicks. Separated daughter templates are formed from the nicked target DNA. Subsequent rounds of nicking and strand displacement synthesis from the target DNA result in linear amplification. An advantage of linear amplification over exponential amplification is that amplified errors in copied strands do not accumulate. Moreover, the rate of amplicon production is sufficient to rapidly accumulate enough DNA for downstream processing.

The term “long” range or “long” target refers to a DNA sequence having a length of at least 300 bases, at least 1 kilobase, at least 5 kilobases, at least 7 kilobases, at least 10 kilobases, at least 20 kilobases, at least 30 kilobases, or at least or up to 50 kilobases.

For example, the length of the target sequence in the target dsDNA may be in the range of 300 bases to 50,000 bases (50 kb).

The term “multiple turnover kinetics” refers to a property of enzymes that bind to substrate, are released from the product of the enzyme reaction and are available to bind to substrate again. (i.e., one molecule of enzyme can produce multiple nicks at different sites on a single day and/or on multiple DNA molecules.

The term “strand specific” refers to the orientation of the recognition sequence where Nb refers to nicking the bottom strand of a DNA duplex with the recognition site in a 5′ to 3′ orientation and Nt refers to the top strand of the duplex.

For example,

Nt.BbvCI

5′-CC*TCAGC-3′

3′-GGAGTCG-5′

Nb.BbvCI

5′-CCTCAGC-3′

3′-GGAGT*CG-5′

Nt.BbvCI (nicking “bottom” relative to “top” of opposite orientation)

5′-GCTGAGG-3′

3′-CGACT*CC-5′

The term “in vitro” refers to a reaction or reactions that are occur in a reaction container. The term “in vitro” is not intended to include a cell or other biological entity.

No primers are required for methods, kits or compositions described herein. Thus, in some embodiments, the methods of the invention are performed in the absence of primers, or the kits and/or compositions of the invention do not comprise primers.

Recognition sites for sequence-specific nickases may be naturally present in the target DNA sequence. Nickases that recognize 5 bases, 6 bases or 7 bases are preferred because the nick sites are spaced out in the target DNA providing for the desired long range amplification between the nick sites.

Alternatively, primers or adaptors may be used to introduce a specific artificial cleavage site or recognition sequence into the target DNA. For example, primers or adaptors that comprise an inosine, uracil, 8-oxoG or ribonucleotide may be used to introduce this site into the target DNA, for site-specific nicking by endonuclease V, UDG and endonuclease VIII, Fpg, or RNaseHII. Primers or adaptors can introduce a recognition sequence for a nickase that is a modified restriction endonuclease, or a guide sequence for guided argonaute cleavage enzyme. Optionally, adaptors may be ligated to the ends of DNA fragments to introduce an artificial nickase recognition site suitable for SDA initiation. In some embodiments, an adaptor comprising a nickase recognition site (e.g. a recognition sequence or artificial cleavage site) is ligated to one or both ends of a target DNA duplex to introduce the nickase recognition site. For example, a loop adaptor having a double-strand stem may be ligated to the end of a duplex where the nickase recognition site such as an inosine, uracil or ribonucleotide is in the stem adjacent to the target DNA fragment.

Various embodiments of the method have several advantages over thermocycling amplification such as PCR or isothermal amplification such as nucleic acid sequence-based amplification (NASBA) that requires a temperature boost to jump start the amplification. Advantages include (a) the ability to achieve amplification at a single temperature; (b) replication of long DNA in a relatively short time (e.g. within about 5 hours, such as within 3 hours); and/or (c) enrichment of the target DNA by as much as 50×-100×enrichment in 3 hours or less at a single temperature such as 37° C. (with normal water bath fluctuations) or any desired temperature in the range of 30° C.-45° C. that avoids errors in sequence that can occur during thermocycling and at high temperatures.

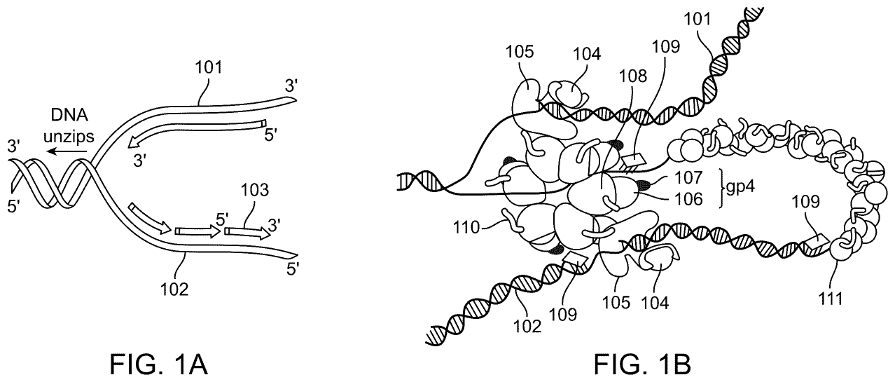

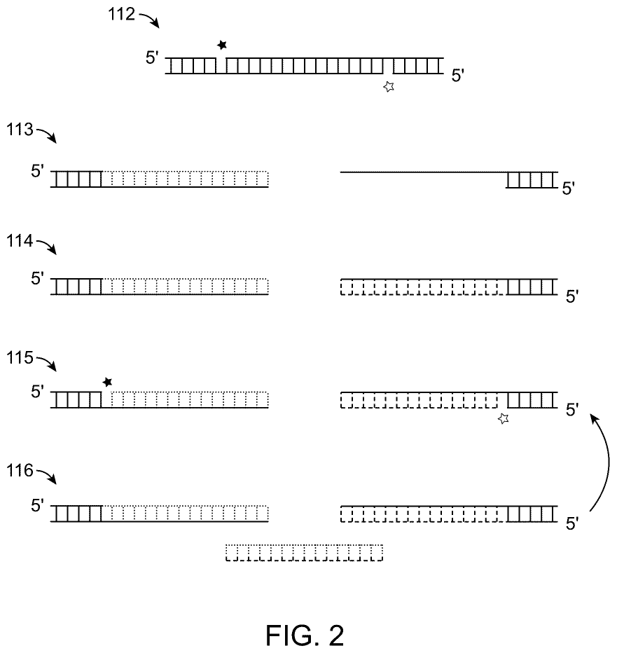

The wild type T7 replisome mechanism for copying duplex DNA is shown in FIG. 1B. It utilizes 4 proteins: a helicase-primase (T7 gp4) and DNA polymerase (T7 gp5), a SSBP (gp2.5) and Trx. These four proteins create a replication fork to duplicate both the leading and lagging strand. The leading strand is copied continuously but the lagging strand is copied in fragments.

In present embodiments, it is preferred to avoid lagging strand synthesis. This is achieved by using a helicase (e.g. gp4) that lacks primase activity responsible for initiating lagging strand synthesis. gp4 consists of two major domains connected by a flexible linker: the C-terminal helicase and the N-terminal primase. The primase domain contains two subdomains: the N-terminal zinc finger binding domain (ZBD) and the RNA polymerase domain (RPD) that are also connected by a flexible linker. In one embodiment, the helicase comprises a mutation within the primase domain that reduces or eliminates primase activity. In one embodiment, the helicase is gp4Δprimase in which the entire primase domain is deleted. Optionally the C-terminal acidic tail (gp4ΔCΔprimase) is also deleted. The helicase domain and the flexible linker connecting helicase with the RPD remains in both circumstances.

Unlike in nature where the replisome is assumed to provide copies of an entire viral genome, the modified replisome is described herein for replicating and thereby amplifying specific target sequences on the leading strand of a DNA sequence within a genome accurately, rapidly and with processivity over a length of up to 50 kb or even longer.

### Nickase

In the present composition, kit and methods, a modified T7 replisome is used with a strand-specific and sequence-specific or site-specific nicking enzyme (nickase). The nickase book marks each end of a target region of the DNA by making a nick on both strands of the duplex. Initiation and extension from one of the DNA nicks will produce a double-stranded break when the nick on the opposite strand is encountered, separating the substrate into two templates for synthesis of complementary strands of the amplicon.

The preferred general properties for a nickase in present compositions, kits and methods is that the nickase be sequence or site-specific, have multiple turnover kinetics and a functional activity at a temperature in the range of 25° C.-42° C. (i.e. mesophilic). Preferably such nickases are active at 37° C. Not all nickases are suited for T7-SDA. For example, unmodified nicking Cas proteins having guide sequences bind to DNA but do not turn over and do not efficiently release nicked DNA product after cleavage. Hence they are not a preferred nickase. Preferred nickases may be selected from: (a) restriction endonuclease derived nickases; (b) an endonuclease that targets a modified base such as uracil (endonuclease III or endonuclease VIII and UDG), inosine (endonuclease V) or 8-oxoG (Fpg); (c) a DNA directed argonaut nicking enzyme (see Vaiskunaite et al. NAR 2022 vol 50 pp 4616-4629); and (d) a repair nuclease, for example, an RNA base and RNase HII that can direct a nick to a specific sequence.

In some embodiments, the nickase is a sequence-specific nickase. Examples of sequence-specific nickases include modified restriction endonucleases that recognize a specific sequence on the DNA and cleave within or at a specified distance outside the recognition sequence. The target DNA sequence (e.g. amplicon for sequencing) preferably has a selected restriction site for nicking at both ends of the target region to be amplified. If this is not the case, a second nickase may be used for obtaining a second nicking event.

There are many nicking endonucleases now available commercially (see for example the New England Biolabs catalog). Generally, a nicking endonuclease retains the cleavage specificity of the restriction endonuclease from which it was derived. Over 200 nicking endonucleases have been described. Many of these have been engineered from restriction endonucleases by inhibiting the cleavage activity on one strand of the DNA. This may be achieved for example, by mutating a subunit in a heterodimeric restriction endonuclease (see for example, U.S. Pat. Nos. 6,191,267 and 7,081,358). Other methods of creating nicking endonucleases from restriction endonucleases have also been described (sec for example U.S. Pat. No. 7,943,303, US 2005/0136462; and US 2008/0268507). Some nicking endonucleases occur naturally such as BstNBI which is a dimer which becomes a monomer on purification because of the weak association of the two subunits. Other modified restriction endonucleases are monomers that recognize and nick one strand and then move to the second strand to form a nick there too (see for example UbaLAI (Sasnauskas, et al, Nucleic Acid Research (2017) 45, 9583-9594)). Because monomers nick sequentially, the one or more nick translating enzymes can initiate nick translation.

Examples of modified restriction endonucleases suitable for use in the present invention include Nb.Bpu10I (CCTNAGC) Nt.Bpu10I (CCTNAGC) Nb.Mva1269I (GAATGC) Nt.Bst9I (GAGTC) Nt.AlwI (GGATC) Nb.BbvCI (CCTCAGC), Nt.BbvCI (CCTCAGC) Nb.BsmI (GAATGC) Nt.BsmAI (GTCTC) Nt.BspQI (GCTCTTC) Nb.BsrDI (GCAATG) Nb.BssSI (CACGAG) Nt.BstNBI (GAGTC) Nb.BtsI. (GCAGTG) Nt.CviPII (CCD) BsoBI (CYCGRG), gHNH (CG/GT) (see for example REBASE® (New England Biolabs, Ipswich, MA)). The recognition sequences for these nickases are provided in parenthesis after each nicking endonuclease where Nb is bottom strand nicking and Nt is top strand nicking.

In embodiments of the invention, the nickase can be any sequence-specific nicking enzyme; preferably one that recognizes 5-7 bases in its recognition sequence. This ensures that the fragment size to be amplified is of reasonable length as the frequency of DNA cleavage by a nickase is inversely proportional to the number of bases in the recognition sequence. Examples and figures provided herein show T7-SDA amplification data using Nt.BbvCI (7 bp recognition sequence, 37° C.), Nb.BbvCI (7 bp recognition sequence, 37° C.). Nb.BssSI (6 bp recognition sequence, 37° C.), Nt.AlwI (5 bp recognition sequence, 37° C.) and Nt.BsmAI (5 bp recognition sequence, 37° C.) all of which can be obtained from New England Biolabs, Ipswich, MA. In some embodiments, the nickase is selected from Nt.BbvCI, Nb.BbvCI and Nb.BssSI.

The amplicon size and general utility of SDA using T7 polymerase (T7 SDA) is limited in part by the specificity of the nickase. A 7 bp recognition sequence that is a feature of Nt.BbvCI and Nb.BbvCI, will appear on average once every 16 kbp in a random DNA sequence.

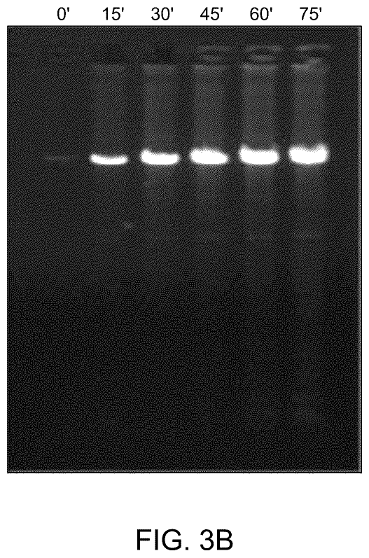

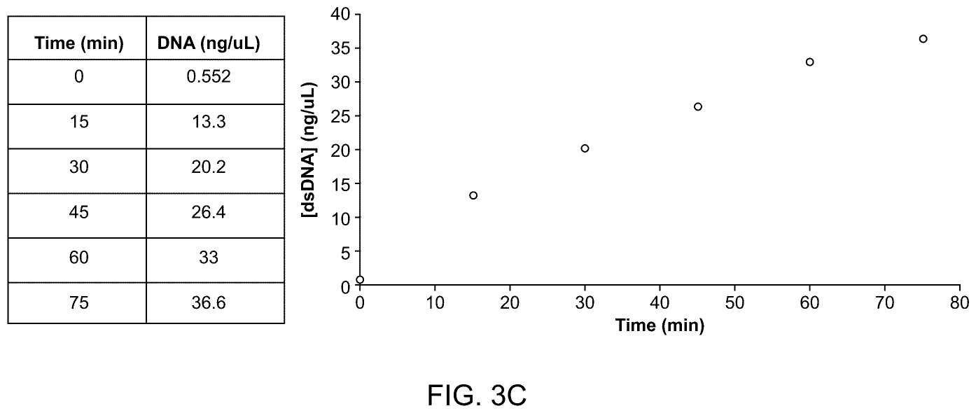

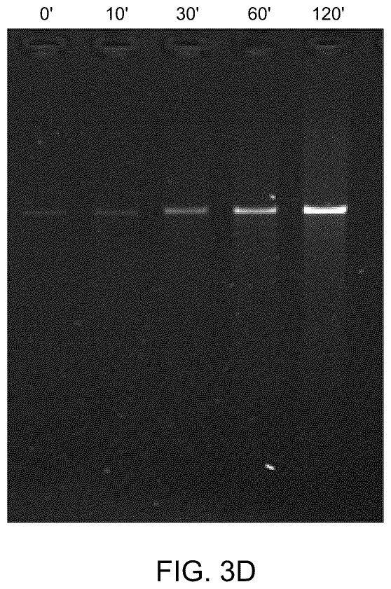

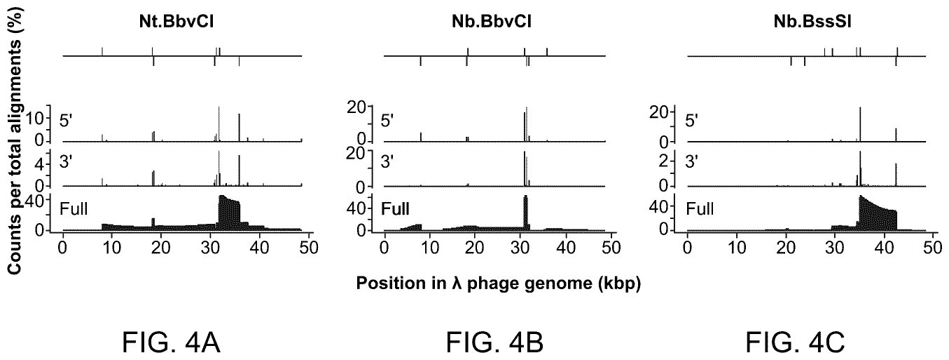

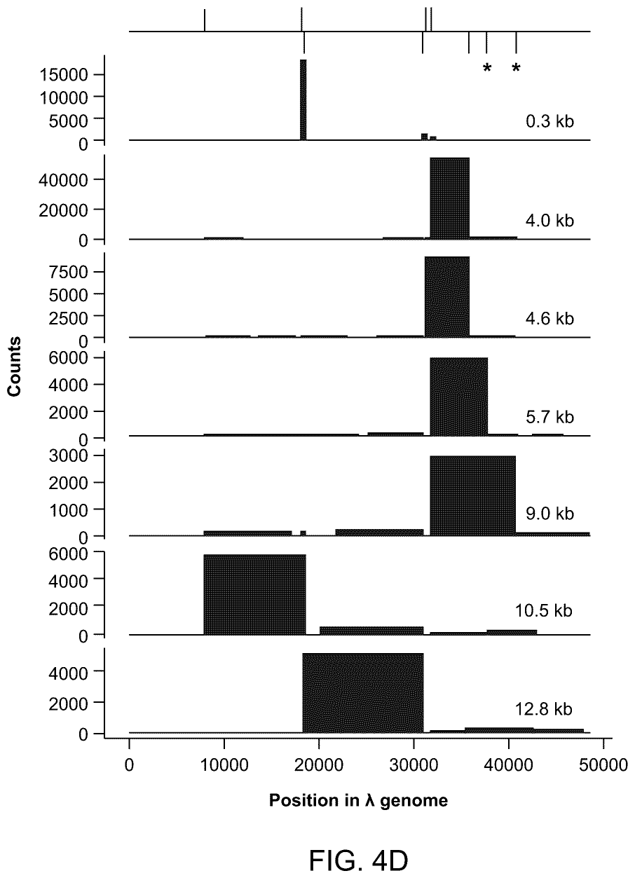

In some embodiments, the nickase is a site-specific nickase that recognizes and targets a modified base, such as uracil, inosine, or 8-oxoG. For example, E. coli endonuclease V is a natural nickase that recognizes damaged nucleotides, with some preference for deoxyinosine, in dsDNA and cleaves the phosphodiester backbone of the harboring strand 3′ of the modified base. Endonuclease V can support repeated rounds of nicking and polymerization. Thus, in some embodiments, the nickase used in the compositions, kits, and methods of the invention is endonuclease V. In FIG. 3D, a 7 kbp substrate containing top and bottom strand deoxyinosine residues was generated by PCR and used as a template in A28 gp5 T7 SDA. Amplification of the expected product was observed by agarose gel electrophoresis.

### Strand Displacing Polymerase

The product of the synthesis of leading strand DNA between 2 sequence-specific or site-specific nick sites is a DNA of defined length. Amplicons correspond to regions of the template between appropriately oriented nicking sites and may be relatively long, up to 23 kbp or as much as 50 kb. In certain embodiments, amplicons hybridize to form an intermediate duplex DNA containing a sequence-specific or site-specific nick site on one strand only of the duplex molecule only. The nicked strand can then be replicated using a strand displacing polymerase that produces complementary DNA in a linear amplification reaction. Because the amplification is linear and copies are repeatedly made from the target DNA leading strand, any errors that occur during copying will be random and different for each copying event. Hence the overall result of linear amplification is enhanced accuracy.

Embodiments of the invention preferably use an exonuclease negative (exo-) T7 DNA polymerase or a variant thereof. The variant may have at least 80%, 85%, or 90% sequence identity with SEQ ID NO:1. A preferred strand displacing polymerase for compositions, kits, methods and uses herein is a genetically engineered form of T7 DNA polymerase (T7 gp5 exo-) that is not affected by secondary structures such as hairpins in substrate DNA when combined with T7 gp4 helicase. The 3′-5′ exonuclease activity can be removed from this polymerase by deletion of 28 amino acids (see for example SEQ ID NO:1) without adversely affecting polymerase activity. A cited disadvantage for the use of exo-T7 DNA polymerase is a possible lack of fidelity. However, it was found previously that the fidelity of copying achieved by exo-T7 DNA polymerase was as much as 5 times better than Taq polymerase (Eckert et al. PCR Methods Appl. 1991 August; 1(1):17-24. doi: 10.1101/gr.1.1.17. PMID: 1842916).

Unlike PCR using Taq polymerase which can only replicate DNA very slowly (e.g., more than 4 hours for typical cycling with a 7 kb fragment), the present method using the highly processive T7 exo DNA polymerase can achieve successful amplification of DNA having sequence fidelity quality that is suitable for sequencing within as little as 15 or 30 minutes (see FIG. 3A-FIG. 3B). The high processivity of the T7 replisome as a whole can synthesize up to 50 kbp of DNA in a single binding event is effective for rapid amplification of long DNA molecules between specified DNA nicks.

### Helicase

Okazaki fragments on the lagging strand of a DNA duplex are undesirable because they form discontinuous, partial copies of the template and impede annealing of the complementary strands of the desired amplicon. Thus, in some embodiments, the composition, kit, and methods of the invention use a helicase that lacks primase activity. This prevents the formation of Okazaki fragments. For example, the helicase (e.g. gp4) may comprise a mutation (e.g. substitution or deletion) within the primase domain that reduces or eliminates primase activity. For example, the helicase may be a modified gp4 helicase that is mutated in (or lacks) the zinc finger binding sub-domain (ZBD) of the primase domain. In one embodiment, the helicase comprises or consists of an amino acid sequence that is at least 80%, 85%, 90%, or 95% identical to gp4ΔCΔprimase (SEQ ID NO:2). For example, the helicase may comprise or consist of SEQ ID NO:2.

In other embodiments, the helicase used in the composition, kit, and methods of the invention has at least some primase activity. In these embodiments, the composition, kit, and methods exclude rCTP and rATP. For example, the composition, kit, and methods may comprise one or more NTPs other than (excluding) rCTP and rATP. The exclusion of ATP and CTP from the method, kit, or composition (e.g. reaction mixture) prevents the formation of undesirable immature Okazaki fragments.

### Single-Strand Binding Protein (SSBP)

The SSBP used in the composition, kit, and methods of the invention may, in some embodiments, be phage T7 SSBP (gp2.5). However, other SSBP may be substituted for gp2.5 such as E.coli SSBP (Spenkelink et al. Nucleic Acids Res 2019 May 7; 47(8):4111-4123. doi: 10.1093/nar/gkz090). Variants of gp2.5 may also be substituted such as described in Hernandez et al. Semin Cell Dev Biol. 2019 February; 86: 92-101.

### Long Range Linear Strand Displacement Amplification

In embodiments, long range linear strand-displacement amplification between 2 nick sites in a target dsDNA (e.g. a genome) using the T7-SDA as described herein for in vitro isothermal amplification technique was analyzed by nanopore sequencing. The reaction steps may be performed in a “one pot” reaction, meaning that at least two of (a), (b) and (c) of the method are performed in a single reaction vessel. For example, (a) and (b), or (b) and (c), or all of (a)-(c) may be performed in the same reaction vessel. In some embodiments, the method is a single step reaction. The method is preferably performed at a temperature in the range of 30° C.-45° C., such as at about 37° C. Preferably the nicking reaction and amplification reaction are performed at about 37° C.

In some embodiments, the composition, kit, and method of the invention use the following components of the T7 replisome: gp2.5 SSBP, modified T7 gp4 lacking primase activity, and 3′-5′ exonuclease negative T7 polymerase (e.g. Sequenase 2.0).

In some embodiments, the results of the method may be analyzed by sequencing, such as by nanopore sequencing. Nanopore sequencing enables sequence determination of individual amplified dsDNA molecules and high-resolution interrogation of T7 SDA.

In the Example described below, the linear 48.5 kb dsDNA λ bacteriophage chromosome was selected as a substrate for reaction with one of three nickases—Nt.BbvCI, Nb.BbvCI and Nb.BssSI. The nickases cut the bottom and top strands of γ phage DNA at multiple positions to enable extension from these nicks by the T7 replisome to produce dsDNA amplicons. As shown in FIG. 2, the modified T7 replisome produces two separated daughter templates from the nicked duplex. The daughter templates are filled in by the T7 DNA polymerase giving rise to a single nick site on each of the daughter templates. Rounds of nicking and strand displacement synthesis result in amplified target DNA.

### The use of Adaptors is Described Above

In some embodiments, the target dsDNA comprises an adaptor containing a nickase recognition site as discussed above. For example, in some embodiments of the methods of the invention, prior to (a), the methods comprise ligating one or more adaptors to a target DNA or to a library of dsDNA comprising the target DNA, wherein the adaptors comprise a recognition site for the nickase; and then (a) comprises nicking the target dsDNA at the nickase recognition site to initiate SDA. For example, the adaptor(s) may comprise a specific sequence that is recognized by a nickase that is a modified restriction endonuclease, or the adaptor(s) may comprise a site (e.g. modified or damaged nucleotide) that is recognized by a site-specific endonuclease, as described herein. In the absence of primers, the nick site or sequence is adjacent to the target nucleic acid sequence. Once the target nucleic acid is nicked, the nick site is not reproduced in the daughter strand but is recreated in the target nucleic acid for further linear amplification (see FIG. 2).

Optionally, primers may be used but are not required for generating amplicons. The use of primers in SDA includes:
(i) Primers designed to complement and hybridize to the sequences immediately 3′ to the nicking endonuclease recognition sequences. ssDNA produced by a round of nicking and extension provides a primer-binding site for production of dsDNA without annealing full complementary strands. Additionally, dsDNA amplicons may have terminal primer binding sites. The primers may invade the duplex DNA with assistance from gp2.5 and gp4; and/or
(ii) Primers designed to include nickase recognition sequences for targeting an arbitrary sequence for amplification. In this case, annealing of the primers to the target sequence, potentially with pre-denaturation of the DNA, may be used to facilitate the first round of amplification from the primers (i.e. these primers may be annealed to the target dsDNA prior to (a)). Subsequent rounds of amplification may then be achieved by the action of the nickase.

### Embodiments

1. A composition comprising: a mesophilic nickase that is strand-specific, sequence-specific and/or site-specific and capable of multiple turnover kinetics, aT7 polymerase (exo-), a helicase, and a single-strand binding protein (SSBP) wherein the composition is free of primers.
2. The composition according to 1, wherein the helicase lacks primase activity.
3. The composition according to any of the above, wherein the helicase has primase activity and wherein the composition comprises NTPs but excludes rCTP and rATP.
4. The composition according to any of the above, wherein the helicase is a modified phage T7 gp4 helicase.
5. The composition according to any of the above, wherein the SSBP is phage T7 (gp2.5).
6. The composition according to any of the above, further comprising E.coli thioredoxin (Trx).
7. The composition of any of the above, wherein the nickase is sequence-specific, wherein the sequence-specific nickase is selected from the group consisting of a modified restriction endonuclease and a mesophilic argonaut having a guide DNA.
8. The composition according to any of the above, wherein the nickase is site-specific wherein the site-specific nickase is selected from the group consisting of endonuclease, III, endonuclease V or endonuclease VIII; formamidopyrimidine DNA glycosylase (Fpg) and RNaseHII.
9. The composition according to any of the above, further comprising a DNA adaptor, wherein the adaptor comprises a nickase specific recognition sequence or a sequence for hybridizing to a guide DNA, or a nick site selected from a ribonucleotide or a modified deoxyribonucleotide such as 8-oxoguanine.
10. The composition according to any of the above, wherein the nickase, T7 polymerase (exo-), SSBP and the helicase are lyophilized or immobilized on a matrix either separately or together.
11. A method for in vitro long range linear amplification of a target double-stranded DNA (dsDNA), comprising:
   (a) combining the dsDNA with the composition of any of claims 1-10 to produce a reaction mix in a reaction container; and
   (b) incubating the reaction mix under isothermal conditions to produce an amplification product.
12. The method according to paragraph 11, wherein in the reaction mix:
   (a) the target dsDNA is nicked by the nickase;
   (b) the amplifying is by leading strand displacement by the DNA polymerase, wherein the individual strands of the dsDNA start at one nick site on one strand and terminate at the nick site on the second strand to produce daughter strands; and
   (c) the daughter strands becoming nicked, thereby permitting rounds of nicking and strand displacement.
13. A method for in vitro long range linear amplification of a target double-strand DNA (dsDNA), comprising:
   (a) nicking the target dsDNA by means of a strand-specific or site-specific mesophilic nickase capable of multiple turnover kinetics;
   (b) amplifying by leading strand displacement with a strand displacing exonuclease negative DNA polymerase in the absence of primers, the individual strands of the dsDNA starting at one nick site on one strand and terminating at the nick site on the second strand to produce daughter strands; and
   (c) permitting rounds of nicking and strand displacement of the target dsDNA for linear amplification.
14. The method according to any of paragraphs 11-13, wherein the strand-specific nickase in (a) is sequence-specific comprising a modified restriction endonuclease having a specific recognition sequence of 5, 6 or 7 bases.
15. The method according to any of paragraphs 11-13, wherein the strand-specific nickase is site-specific nickase comprising an endonuclease selected from the group consisting of endonuclease III, V, and VIII.
16. The method of any of paragraphs 11-15, wherein the helicase lacks primase activity or wherein rCTP and rATP are excluded from the method.
17. The method according to any of paragraphs 11-16, wherein the polymerase is a T7 polymerase (exo-).
18. The method according to any of paragraphs 11-17, further comprising hybridizing one or more primers to a DNA sequence in the target DNA or daughter strands that is 3′ adjacent to the nickase recognition site or sequence.
19. The method according to any of paragraphs 11-18, further comprising hybridizing one or more primers to the target DNA wherein the primers contain a universal sequence for hybridization to the target DNA.
20. The method according to any of paragraphs 11-19, wherein (a) further comprises: ligating adaptors to a library of dsDNA, wherein the adaptors contain a nickase recognition site and optionally primer binding sites.
21. The method according to any of paragraphs 11-20, comprising nicking the target dsDNA at the nick site to initiate strand displacement amplification.
22. The method according to any of paragraphs 11-21, wherein the target dsDNA has a target sequence that has a length in the range of 300 bases to 50,000 bases.
23. The method according to any of paragraphs 13-22, wherein two or more of steps (a), (b), and (c) are performed in the same reaction vessel.
24. The method according to any of paragraphs 13-23, wherein (a)-(c) is a one-step reaction.
25. The method according to any of paragraphs 11-24, wherein the reactions are performed at 37° C.
26. A method for amplifying long target DNA optionally without primers, comprising:
   (a) nicking a target DNA on opposing strands with a strand-specific, sequence-specific or site-specific mesophilic nickase capable of multiple turnover kinetics;
   (b) replicating only the leading strand of the target DNA with a T7 exo-polymerase in the presence of a helicase, a SSBP and dNTPs; and
   (c) producing full length amplicons from a target DNA of at least 300 bases to 50 kilobases.
27. A kit comprising: a strand-specific, sequence-specific or site-specific mesophilic nickase capable of multiple turnover kinetics, T7 polymerase (exo-), a SSBP, a helicase and dNTPs in a single container or in separate containers either in a buffer, immobilized on a matrix or lyophilized.

### General Considerations

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and case of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely.” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′-and 3′-ends (e.g., methylation) of the nucleic acid. A “non-naturally occurring” composition may differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, aqueous; and (c) having one or more additional components beyond those found in nature (e.g., buffering agents, a detergent, a dye, a solvent or a preservative).

All publications, patents, and patent applications mentioned in this specification are herein incorporated by reference to the same extent as if each individual publication, patent, or patent application was specifically and individually indicated to be incorporated by reference including U.S. 63/208,534 and U.S. 63/208,621.

### EXAMPLES

Materials. Except for Sequenase v2.0 (428 gp5, Thermo Fisher, Waltham, MA) and unless otherwise noted, all enzymes and reagents are available from New England Biolabs, Ipswich, MA or purified as described.

Purification of T7 gp4 and gp2.5. This was performed using standard techniques (see for example: Mendelman et al. J Biol Chem. 1991, 266 (34):23240-50; Perler, et al. Proc Natl Acad Sci. 1992; 89(12):5577-81; Dunn et al. J Mol Biol. 1983:166(4):477-535; Rosenberg et al. J Biol Chem. 1992; 267(21):15005-12 and Rezende et al. J Biol Chem. 2002; 277(52):50643-53).

Reaction of λ DNA with the T7 replisome and nicking enzymes. Genomic λ phage DNA (100 ng) was incubated with components of the T7 replisome and a nickase in reaction buffer (50 mM Tris acetate pH 7.9, 50 mM potassium acetate, 2 mM DTT, 3.5 mM dTTP, 1 mM other dNTPs). Components of the T7 replisome include gp2.5 (5 μM dimer), gp4 (100 nM hexamer) and either gp5-Trx (200 U mL⁻¹, 80 nM) or Sequenase 2.0 (200 U mL⁻¹). The nicking endonucleases (200 U mL⁻¹) used in this study are Nb.BssI, Nb.BbvCI and Nt.BbvCI). Reactions (50 μL) were initiated with 10 mM magnesium acetate, incubated at 37° C. for 3 hours, and quenched with a mixture of 20 mM EDTA and proteinase K. Portions of the reactions were visualized by non-denaturing or alkaline agarose gel electrophoresis, and the remainder were prepared for nanopore sequencing.

Nanopore sequencing. Quenched amplification reactions were purified using Ampure® XP magnetic beads with two washes of 70% ethanol (Beckman Coulter, Brea, CA) and eluted in nuclease-free H₂O (nfH₂O, Thermo Fisher, Waltham, MA). DNA concentrations were quantified using a Qubit® fluorimeter (Molecular Probes, Eugene, OR) and the 1×dsDNA High Sensitivity (HS) Kit (Thermo Fisher, Waltham, MA). Each reaction was end-repaired using the NEBNext® Ultra™ II End Repair/dA-Tailing Module (New England Biolabs, Ipswich, MA) prior to barcode ligation. A 50 μL solution of 1 μg DNA was combined with 7 μL of the buffer mix and 3 μL of the enzyme mix, incubated at 20° C. for 5 minutes and then 65° C. for 5 minutes. Barcode oligos from the Native Barcoding Kit (2.5 μL, Oxford Nanopore Technologies (ONT), Oxford, UK) were added to the repaired libraries along with components of the NEBNext Ultra II Ligation Module (1.5 μL ligation enhancer, 34.5 μL master mix, 1.5 μL nfH₂O, New England Biolabs, Ipswich, MA) and ligation occurred in 10 minutes at room temperature. Barcoded libraries were purified using magnetic beads, quantified and pooled in approximately equal proportion by mass.

Adaptor ligation for ONT sequencing was accomplished using the Ligation Sequencing Kit (New England Biolabs, Ipswich, MA). Pooled barcoded libraries (1 μg) were combined to 100 μL with 5 μL barcode adaptor mix, 20 μL Quick Ligation Reaction Buffer (New England Biolabs, Ipswich, MA), and 10 μL Quick T4 DNA Ligase in nfH₂O before incubating on the bench for 10 minutes. The final library was purified using magnetic beads and two washes with LFB buffer before elution into EB buffer (ONT) and quantification by Qubit. ONT sequencing was performed using a GridION® sequencer (ONT, Oxford, UK), according to the recommendations of the manufacturer. Basecalling and demultiplexing was performed using the default settings of the MinKNOW® software suite (ONT, Oxford, UK).

### Results

We first set out to demonstrate SDA using the T7 replisome with a nickase that was a sequence-specific nicking endonuclease (nickase). Genomic λ phage DNA was selected as a substrate because its size (48.5 kbp) enables robust sequencing depth of reaction products while presenting numerous sites for amplification initiation by available nickases. Nickases selectively cut either the top or bottom strand of a dsDNA molecule. The endonucleases Nt.BbvCI and Nb.BbvCI create either a top or bottom strand nick, respectively, at the recognition sequence 5′-CCTCAGC, which occurs seven times in the λ phage genome (Sanger F, et al. J Mol Biol. 1982; 162(4):729-73.8). The nicking enzyme Nb.BssSI created a bottom strand nick at three different 5′-CACGAG sites and a top strand nick at five 5′-CTCGTG sites. Each nicking enzyme produced varied patterns of initiation sites for the T7 replisome within the λ phage DNA substrate. This resulted in distinct bands of dsDNA amplicons generated by cycles of nicking and DNA polymerization by the T7 replisome (FIG. 3A and FIG. 4A-FIG. 4C).

In reactions using the exonuclease deficient T7 replisome, amplicons were tentatively assigned according to a simple amplification scheme (FIG. 2) and the position of nicking recognition sequences in λ phage DNA. For instance, the principal amplicon in the reaction with Nb.BssSI is about 7 kbp in length. This corresponds to a region between top-strand nicking site ³⁵²¹⁹CTCGTG and bottom-strand site ⁴²⁴¹⁶CACGAG. Similarly, the ˜4 kbp band in the reaction with Nt.BbvCI corresponds to amplification of the region between top-strand ³¹⁸³⁶CCTCAGC and bottom-strand ³⁵⁸¹³GCTGAGG sites. Reactions using the WT T7 replisome produce different amplicons compared to those using the exonuclease deficient polymerase.

Nanopore sequencing of T7 products. To assign the discrete products apparent in the agarose gel of the SDA reactions, and to elucidate the role of gp5 exonuclease activity in the reaction, we employed nanopore sequencing. Nanopore sequencing yielded sequence reads for each of the six T7 reactions shown in FIG. 3A. When Δ28 gp5 polymerase was used, clusters of sequencing read lengths corresponded to visible bands on the agarose gel. For example, the 7 kbp amplicon produced with Nb.BssSI generated nanopore data showing clustering of sequencing read lengths of 7 kilonucleotides (knt). The sequencing reads could be aligned to λ phage DNA using the minimap2 algorithm (Table 1).

TABLE 1
Nanopore sequencing statistics for T7 SDA reactions.

| | Nt.BbvCI | Nt.BbvCI | Nb.BbvCI | Nb.BbvCI | Nb.BssSI | Nb.BssSI |
|---|---|---|---|---|---|---|
| Polymerase | Δ26 gp5 | gp5 | Δ26 gp5 | gp5 | Δ26 gp5 | gp5 |
| Reads | 317083 | 753050 | 457596 | 953507 | 210457 | 192085 |
| Read Length N50 | 5377 | 6986 | 7878 | 9303 | 7106 | 7098 |
| Q > 10 Reads^{b} | 234538 | 498176 | 294340 | 651211 | 137052 | 101687 |
| Unaligned Reads | 2013 | 26846 | 1037 | 2266 | 1210 | 563 |
| Alignments | 351132 | 877706 | 523911 | 252373 | 258166 | 256629 |
| Sup. Alignments^{c} | 36062 | 151502 | 67352 | 155232 | 48919 | 65107 |
| Chimeric Reads^{d} | 32627 | 128674 | 60588 | 128859 | 43977 | 54683 |

^{b}Reads with average basecalling quality score over 10.
^{c}Number of supplementary alignments found by minimap2
^{d}Determined by counting the number of reads with at least one supplementary alignment.

An example of a coverage map of the 3′ and 5′ ends of the alignments, as well as full alignment intervals, are shown in FIG. 4A-FIG. 4C for Nt.BbvCI, Nb.BbvCI and Nb.BssSI with Δ26 gp5. This coverage map and others for other nickases tested, form the basis for a mechanistic understanding of the T7 SDA reaction. The position and orientation of nickase recognition sites is shown on top of the histograms. These broadly correspond to the 5′ and 3′ termini of alignments. 5′ ends were generated in the SDA reaction by the nicking enzyme and had lower heterogeneity compared to 3′ ends which result from polymerase activity. For Nt.BbvCI, the 5′ coverage map revealed several off-target sites that differed from the cognate recognition sequence by a single nucleotide: ²⁰¹⁴⁵GCTCAGG, ⁴⁰⁷⁹⁸GCTTAGG and ³⁷⁵⁸⁶GCTTAGG. The 5′ coverage maps demonstrated that amplified dsDNA was generated by repeated cycles of nicking and strand-displacing DNA polymerization. Reactions with the exonuclease deficient T7 replisome showed prominent regions of high coverage between sequential top and bottom-strand nicking recognition sites as expected from the amplification scheme of FIG. 2 (first top nick site to first bottom nick site, first bottom nick site to second top nick site etc.).

Identification of dsDNA amplicons in the reaction of Δ28 gp5 T7 replisome and Nt.BbvCI is demonstrated in FIG. 4D. The dominant 4 kbp amplicon aligned as anticipated between sequential top and bottom strand Nt.BbvCI recognition sites. Longer amplicons up to 13 kbp corresponded to amplification between initiation sites with one intervening nicking recognition site. The longest amplification product was a single 23 knt read that aligns as a reverse complement between ⁸⁰¹⁴T and ³⁰⁹²⁰A, covering two Nt.BbvCI recognition sequences. These amplicons represent the prominent bands on the agarose gel (FIG. 3A) produced by the exonuclease deficient T7 replisome.

What is claimed is:

1. A composition comprising: a mesophilic nickase that is strand-specific, sequence-specific and/or site-specific and capable of multiple turnover kinetics, aT7 polymerase (exo-), a helicase, and a single-strand binding protein (SSBP) wherein the composition is free of primers.

2. The composition according to claim 1, wherein the helicase lacks primase activity.

3. The composition according to claim 1, wherein the helicase has primase activity and wherein the composition comprises NTPs but excludes rCTP and rATP.

4. The composition according to claim 2, wherein the helicase is a modified phage T7 gp4 helicase.

5. The composition according to claim 1, wherein the SSBP is phage T7 (gp2.5).

6. The composition according to claim 1, further comprising E.coli thioredoxin (Trx).

7. The composition of claim 1, wherein the nickase is sequence-specific, wherein the sequence-specific nickase is selected from the group consisting of a modified restriction endonuclease and a mesophilic argonaut having a guide DNA.

8. The composition claim 1, wherein the nickase is site-specific, wherein the site-specific nickase is selected from the group consisting of endonuclease, III, endonuclease V or endonuclease VIII: formamidopyrimidine DNA glycosylase (Fpg) and RNaseHII.

9. The composition according to claim 1, further comprising a DNA adaptor, wherein the adaptor comprises a nickase specific recognition sequence or a sequence for hybridizing to a guide DNA, or a nick site selected from a ribonucleotide or a modified deoxyribonucleotide such as 8-oxoguanine.

10. The composition according to claim 1, wherein the nickase, T7 polymerase (exo-), SSBP and the helicase are lyophilized or immobilized on a matrix either separately or together.

11. A method for in vitro long range linear amplification of a target double-stranded DNA (dsDNA), comprising:
(a) combining the dsDNA with the composition of claim 1 to produce a reaction mix: and
(b) incubating the reaction mix under isothermal conditions to produce an amplification product.

12. The method of claim 11, wherein in the reaction mix:
(a) the target dsDNA is nicked by the nickase:
(b) the amplifying is by leading strand displacement by the DNA polymerase, wherein the individual strands of the dsDNA start at one nick site on one strand and terminate at the nick site on the second strand to produce daughter strands: and
(c) the daughter strands becoming nicked, thereby permitting rounds of nicking and strand displacement.

13. A method for long range linear amplification of a target double-strand DNA (dsDNA), comprising:
(a) nicking the target dsDNA by means of a strand-specific or site-specific mesophilic nickase capable of multiple turnover kinetics:
(b) amplifying by leading strand displacement with a strand displacing, exonuclease negative, DNA polymerase in the absence of primers, the individual strands of the dsDNA starting at one nick site on one strand and terminating at the nick site on the second strand to produce daughter strands; and
(c) permitting rounds of nicking and strand displacement on the target dsDNA for linear amplification.

14. The method according claim 11, wherein the strand-specific nickase in (a) is sequence-specific comprising a modified restriction endonuclease having a specific recognition sequence of 5, 6, or 7 bases.

15. The method according claim 11, wherein the strand-specific nickase is site-specific nickase comprising an endonuclease selected from the group consisting of endonuclease III, V, and VIII.

16. The method of claim 11, wherein the helicase lacks primase activity or wherein rCTP and rATP are excluded from the method.

17. The method according to claim 11, wherein the polymerase is a T7 polymerase (exo-).

18. The method according to claim 11, further comprising hybridizing one or more primers to a DNA sequence in the target DNA or daughter strands that is 3′ adjacent to the nickase recognition site or sequence.

19. The method according to claim 11, further comprising hybridizing one or more primers to the target DNA wherein the primers contain a universal sequence for hybridization to the target DNA.

20. The method according to claim 11, wherein (a) further comprises: ligating adaptors to a library of dsDNA, wherein the adaptors contain a nickase recognition site and optionally primer binding sites.

21. The method according to claim 20, comprising nicking the target dsDNA at the nick site on the adaptors to initiate strand displacement amplification (SDA).

22. The method according to claim 11, wherein the target dsDNA has a target sequence for amplification that has a length in the range of 300 bases to 50,000 bases.

23. The method according to claim 13, wherein two or more of steps (a), (b), and (c) are performed in the same reaction vessel.

24. The method according to claim 13, wherein (a)-(c) is a one-step reaction.

25. The method according to claim 11, wherein the reactions are performed at 37° C.

26. A method for amplifying long target DNA optionally without primers, comprising:
(a) nicking a target DNA on opposing strands with a strand-specific, sequence-specific or site-specific mesophilic nickase capable of multiple turnover kinetics;
(b) replicating only the leading strand of the target DNA with a T7 exo-polymerase in the presence of a helicase, a single-strand binding protein (SSBP) and dNTPs; and
(c) producing full length amplicons from a target DNA of at least 300 bases to 50 kilobases.

27. A kit comprising: a strand-specific, sequence-specific or site-specific mesophilic nickase capable of multiple turnover kinetics, T7 polymerase (exo-), a single-strand binding protein (SSBP), a helicase and dNTPs in a single container or in separate containers either in a buffer, immobilized on a matrix or lyophilized.


SEQUENCE LISTING (2 sequences)

SEQ ID NO: 1 - protein, 564 aa, Artificial Sequence
  Synthetic construct
DNSHDSDSVFLYHIPCDNCGSSDGNSLFSDGHTFCYVCEKWTAGNEDTKERASKRKPSGGKPGTYNVWNFGESNGRYSALTARGISKETCQKAGYWIAKVDGVMYQVADYRDQNGNIVSQKVRDKDKNFKTTGSHKSDALFGKHLWNGGKKIVVTEGEIDMLTVMELQDCKYPVVSLGHGASAAKKTCAANYEYFDQFEQIILMFDMDEAGRKAVEEAAQVLPAGKVRVAVLPCKDANECHLNGHDREIMEQVWNAGPWIPDGVVSALSLRERIREHLSSEESVGLLFSGCTGINDKTLGARGGEVIMVTSGSGMGKSTFVRQQALQWGTAMGKKVGLAMLEESVEETAEDLIGLHNRVRLRQSDSLKREIIENGKFDQWFDELFGNDTFHLYDSFAEAETDRLLAKLAYMRSGLGCDVIILDHISIVVSASGESDERKMIDNLMTKLKGFAKSTGVVLVVICHLKNPDKGKAHEEGRPVSITDLRGSGALRQLSDTIIALERNQQGDMPNLVLVRILKCRFTGDTGIAGYMEYNKETGWLEPSSYSGEEESHSESTDWSNDTD

SEQ ID NO: 2 - protein, 502 aa, Artificial Sequence
  Synthetic construct
TYNVWNFGESNGRYSALTARGISKETCQKAGYWIAKVDGVMYQVADYRDQNGNIVSQKVRDKDKNFKTTGSHKSDALFGKHLWNGGKKIVVTEGEIDMLTVMELQDCKYPVVSLGHGASAAKKTCAANYEYFDQFEQIILMFDMDEAGRKAVEEAAQVLPAGKVRVAVLPCKDANECHLNGHDREIMEQVWNAGPWIPDGVVSALSLRERIREHLSSEESVGLLFSGCTGINDKTLGARGGEVIMVTSGSGMGKSTFVRQQALQWGTAMGKKVGLAMLEESVEETAEDLIGLHNRVRLRQSDSLKREIIENGKFDQWFDELFGNDTFHLYDSFAEAETDRLLAKLAYMRSGLGCDVIILDHISIVVSASGESDERKMIDNLMTKLKGFAKSTGVVLVVICHLKNPDKGKAHEEGRPVSITDLRGSGALRQLSDTIIALERNQQGDMPNLVLVRILKCRFTGDTGIAGYMEYNKETGWLEPSSYSGEEESHSESTDWSNDTDF